(19) United States
(12) Patent Application Publication
(10) Pub. No.: US 2003/0192958 A1
(43) Pub. Date: Oct. 16, 2003

(54) Floral arrangement set in a bed containing a volatile air fragrance compound

(76) Inventor: Haruo Miyagi, Lehi, UT (US)

Correspondence Address: Angus C. Fox, III, 4093 N. Imperial Way, Provo, UT, 84604-5386, US

(21) Appl. No.: 10/121,364

(22) Filed: Apr. 12, 2002

(51) Int. Cl. A61L 9/04

(52) U.S. Cl. 239/6; 239/60; 239/211

(57) ABSTRACT

A decorative air fragrance dispenser includes an open container having a base portion and a perimetric wall portion continuous with the base portion, a floral arrangement anchoring device positioned within the container; a floral arrangement secured to the floral arrangement anchoring device, and a sublimatable aromatic compound covering the floral arrangement anchoring device and providing adhesion between the floral arrangement anchoring device and said vessel. The floral anchoring device may be a collapsible polymeric foam block, multiple sandwiched layers of wire mesh, a pinpoint holder, or a block of compressed wood particles that has been drilled or pierced to accept floral stems. The floral arrangement may include artificial flowers or dried natural flowers. The sublimatable aromatic compound may be a gel.

### BACKGROUND OF THE INVENTION

1. Field of the Invention

This invention relates to air fragrance dispensers and deodorizers and, more specifically, to displays of natural or artificial flowers which are combined with a fragrance dispensing device.

2. Description of the Prior Art

For more than a century, many inventions have been directed to methods for dispensing fragrances into the air. For example, U.S. Pat. No. 355,982, which issued to William Eggert, Jr. on Jan. 11, 1887, discloses an artificial flower having a hollow stem which draws perfume from a reservoir via capillary action. The heart, or central portion of the flower, which is absorbent, is the recipient of the perfume withdrawn from the reservoir. The absorbent heart has an area sufficient to facilitate evaporation of the perfume into the surrounding ambiance. A similar product is disclosed in U.S. Pat. No. 3,400,890, which issued to Francis E. Gould on Sep. 10, 1968. Hydrophillic polymeric material is used to fabricate the petals and stamen of an artificial flower. An absorbent wick is employed to transport perfume from a reservoir to the flower parts for evaporation into the air. Another similar product is disclosed in U.S. Pat. No. 4,708,851, which issued to Andreas Freytag Von Loringhoven on Nov. 24, 1987. The disclosed article is a wick or fleece-like tampon impregnated with a perfume, which can serve as the stamen of an artificial flower. Still another example is U.S. Pat. No. 1,886,429, which issued to Alvin L. Saeks on Nov. 8, 1932. This patent discloses an artificial flower that has been dipped in molten or solvated naphthalene. Coloring and perfume may be added to the naphthalene to give the artificial flower a desired scent and color. An even more recent patent, U.S. Pat. No. 5,353,546, which issued to Ronald F. Bock on Oct. 11, 1994, discloses a combination vase and air fragrance dispenser comprised of a pair of nested vessels, one to hold natural or artificial flowers, the other to hold a solid or liquid air treatment material.

Aromatic compounds used as air freshener compounds are generally volatile and flammable. If such a compound, whether a solid or a liquid, is freestanding in an open container, smokers may be tempted to extinguish their cigarettes in the container. Even an infrequent use in such a manner may create an unacceptable fire hazard.

In addition, there is a need for an air fragrance dispenser which has sufficient surface area so as not to require a powered fan to move air across the surface of the aromatic compound. Without some decorative disguise, there would be little to differentiate such an article from the deodorizer blocks used in toilets and urinals.

### SUMMARY OF THE INVENTION

The present invention provides an air fragrance dispenser that is both decorative and likely to discourage improper use. The new dispenser includes an open container or vessel, a floral arrangement anchoring device positioned within the container; a floral arrangement secured to the anchoring device, and a sublimatable aromatic compound at least partially covering the anchoring device and providing adhesion between the floral arrangement anchoring device and the container. The floral anchoring device may be a collapsible polymeric foam block, multiple sandwiched layers of wire mesh, a pinpoint holder, a block of wood or a block of compressed wood particles that has been drilled or pierced to accept floral stems. The floral arrangement may include artificial flowers, dried natural flowers and other natural or artificial foliage. The sublimatable aromatic compound may be a gel, or it may be a compound that is a solid having a melting point greater than about 40° C., the maximum likely room temperature.

The invention also provides a method of making an air fragrance dispenser. The method includes the steps of providing an open vessel of a desired size and shape; providing a plurality of arrangeable floral items; providing a floral arrangement holder; providing a liquified aromatic deodorizer compound; attaching each floral item to the floral arrangement holder; placing the floral arrangement holder and attached floral items within said vessel; pouring the liquified aromatic deodorizer compound into the vessel until the floral arrangement holder is at least partially submerged; and allowing the liquified aromatic deodorizer compound to solidify.

### DESCRIPTION OF THE DRAWINGS

FIG. 1 is an isometric view of an open container;

FIG. 2 is a side elevational cut-away view of the open container of FIG. 1;

FIG. 3 is an isometric view of a collapsible styrofoam block floral arrangement holder;

FIG. 4 is a side elvevational view of the floral arrangement holder following the installation of floral elements therein;

FIG. 5 is a side elevational cut-away view of the open container of FIG. 1, following placement therein of the floral arrangement holder and attached foral elements;

FIG. 6 is a side elevational cut-away view of the assembly of FIG. 4, after a liquified aromatic deodorizer compound has been poured over the floral arrangement holder, covering it completely, and allowed to subsequently solidify;

FIG. 7 is a plan view of a rectangular piece of wire mesh suitable for forming a mesh floral arrangement holder;

FIG. 8 is a piece of mesh like that shown in FIG. 7 that has been folded over itself multiple times for form a wire mesh sandwich floral arrangement holder;

FIG. 9 is an isometric view of a drilled or pierced block made of wood or compressed wood particles;

FIG. 10 is a side elevational view of a pinpoint floral arrangement holder; and

FIG. 11 is a side elevational cut-away view of an assembly which includes the floral arrangement of FIG. 4, without the holder, set in a bed consisting entirely of a aromatic deodorizer compound that has been allowed to solidify within the open container of FIG. 1.

### DETAILED DESCRIPTION OF THE INVENTION

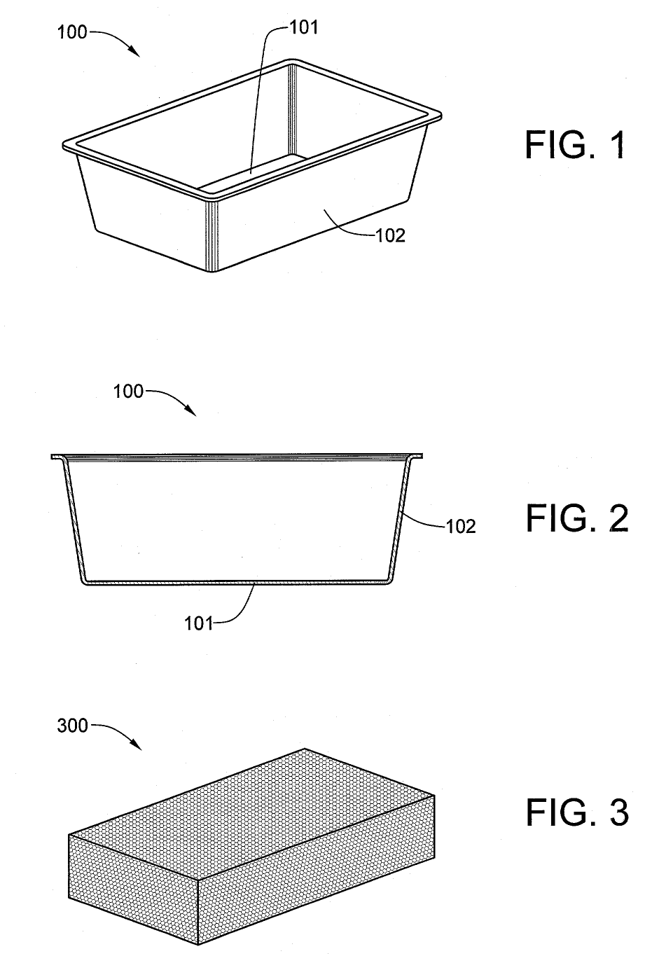

The invention and its method of manufacture will now be described in detail with reference to the attached drawing FIGS. 1 through 10.

Referring now to FIG. 1, a flower box 100 is a generally rectangularly-shaped open container having a base portion 101 and a perimetric wall portion 102. It should be understood that the invention contemplates the use of any open container of suitable size, and may include flower boxes constructed of metal, plastic, and wood; clay, stone, ceramic, and porcelain flower pots; and glass, plastic, ceramic and stone vases, particularly of the wide-mouth variety.

Referring now to FIG. 2, thw flower box 100 of FIG. 1 is shown in a cross-sectional view taken through a vertical plane which longitudinally bisects the box.

Referring now to FIG. 3, a generally rectangular block 300 of collapsible, polymeric floral foam is shown. Such material, which is marketed under the brand names of Fill-Fast-Foam®, Bar-Fast®, Oasis® and Quickee®, is an excellent holder for dried plant materials and artificial flowers and leaves which hagve stiff wire stems. The foam material is easily cut into desired shapes. For hard stemmed items, the foam may be punctured directly. However, for soft stemmed and delicate items, the foam must be pierced with a awl or some similar tool and the lower end of the stem inserted within the hole. As floral foam is typically an open-pore material, it readily absorbs water and other liquids.

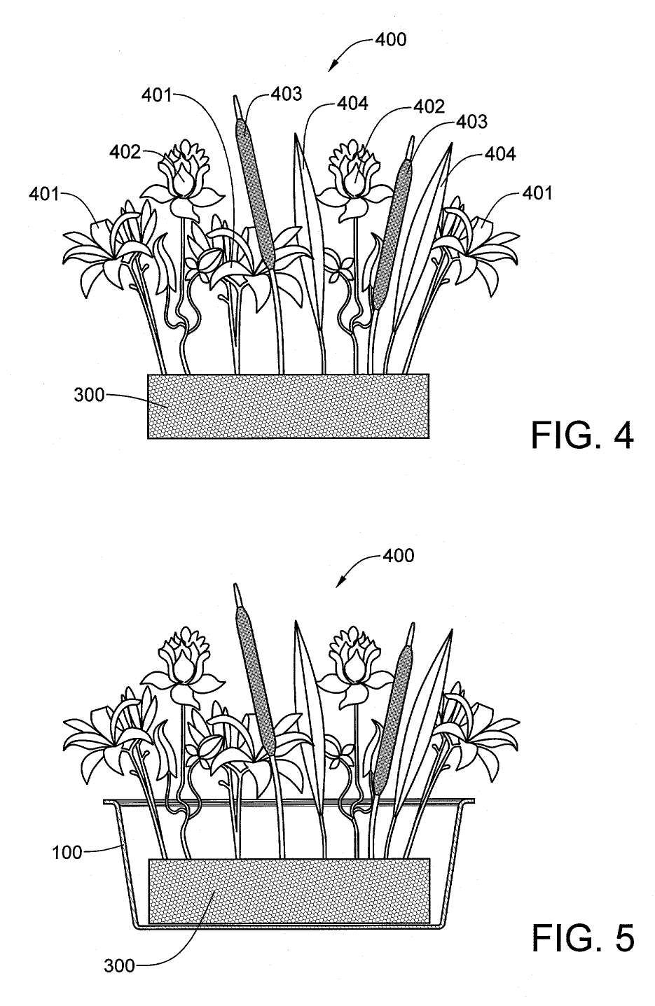

Referring now to FIG. 4, a number of foliage items have been inserted at one end into the collapsible floral foam block 300 to form a floral, or foliage arrangement 400. The arrangement includes lilies 401, lotus flowers 402, cattails 403, and leaves 404. Other items, such as grain stalks, fern fronds, twigs, moss, and small branches are to be considered material from which such a foliage or floral arrangement may be made.

Referring now to FIG. 5, the floral arrangement 400 and the foam block 300, which serves as a floral arrangement holder, have been inserted within the flower box 100.

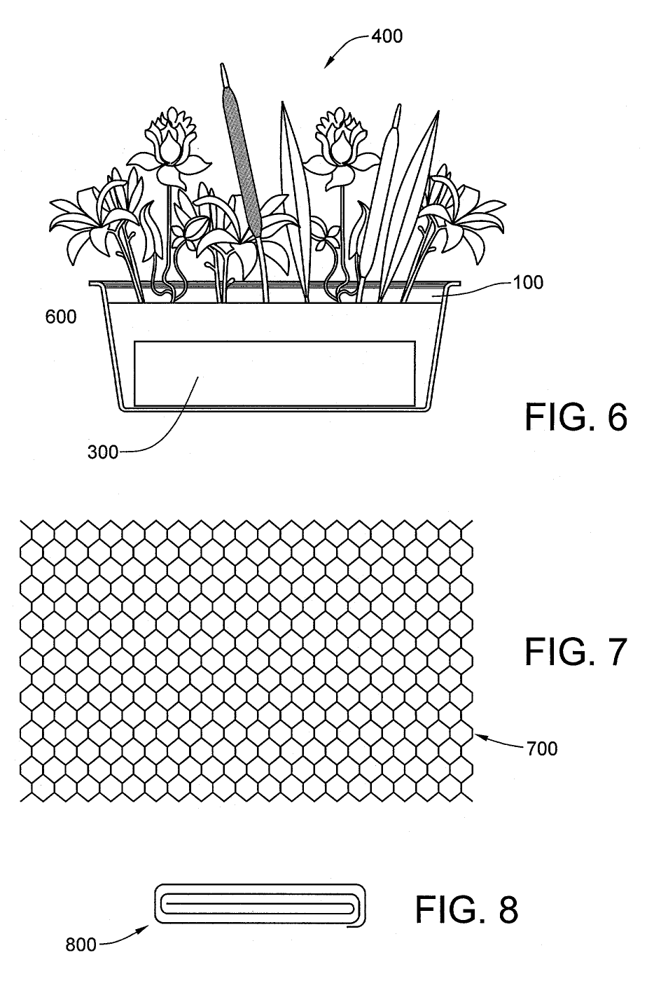

Referring now to FIG. 6, a liquified aromatic deodorizer compound 600 has been poured over the floral arrangement holder. Two types of deodorizer compound are contemplated by this invention. The first is a solid material at, and at least slightly above room temperature. For example, a solid deodorizer compound that melts at a temperature greater than 40° C. is likely to remain in a solid condition, even if left in direct sunlight for extended periods of time. The second type of deodorizer compound is a gel material that solidifies through a coagulation process. In any case, once the deodorizer compound has solidified, the flower box and the floral or foliage arrangement 400 may be handled without risk of spilling the deodorizer compound 600.

Referring now to FIG. 7, a rectangular piece of wire mesh 700 having the same configuration as chicken wire, is shown. Such a piece of mesh may be used to fabricate a floral arrangement holder. Whereas chicken wire is typically a 2.5 cm mesh, the ideal mesh size for this application is about 1.0 cm. Available with a green or brown enameled coating, it is suitable for forming a multi-layer mesh floral arrangement holder.

Referring now to FIG. 8, a rectangular piece of wire mesh, such as the mesh 700 shown in FIG. 7 has been folded folded on itself several time, resulting in a wire mesh sandwich floral arrangement holder 800 that is five layers thick. This wire mesh floral arrangement holder 800 may be substituted for the floral foam flower arrangement holder 300. It is particularly well-suited for mass arrangements in vases or deep bowls. When constructing the floral or foliage arrangement, each stem should pass through at least three layers of meshes or wires.

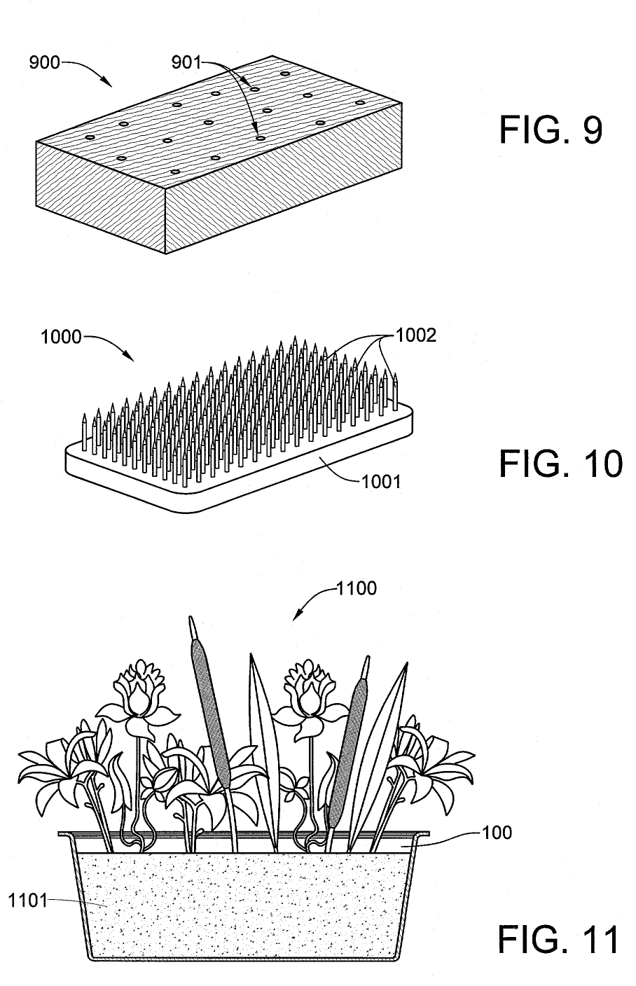

Referring now to FIG. 9, a block of wood, or a block of compressed wood particles 900 has been drilled or pierced to form a floral arrangement holder. Individual floral or foliage items may be inserted within the holes 901 formed by drilling or piercing. This drilled or pierced wood, or compressed wood block 900 may be substituted for the floral foam flower arrangement holder 300.

Referring now to FIG. 10, a rectangularly-shaped pinpoint floral arrangement holder 1001 having a base member 1001 and multiple pins 1002 embedded within the base member 1001, is shown. This pinpoint holder 1000 may be substituted for the floral foam flower arrangement holder 300. Pinpoint holders are particularly suited for line or line-mass arrangements in low bowls or shallow pedestal containers. Stems are stuck directly onto the pins or are wdeged between them. Several thin stems may be bound together with string or tape.

Referring now to FIG. 11, an alternative embodiment of the invention includes

FIG. 11 is a side elevational cut-away view of an alternative assembly 1100, which includes the open container 100 of FIG. 1, the foliage arrangement 400 of FIG. 4, and an aromatic deodorizer compound 1101, which partially fills the open container 100. The foliage arrangement 400, the stalks and stems of which have been embedded within the aromatic deodorizer compound 1101 that is either a partially coagulated gell or a solid material that has been partially softened by heating. After the foliage arrangement 400 is properly positioned within the aromatic deodorizer compound 1101, the aromatic deodorizer compound 1101 is allowed to completely solidify.

Although only several embodiments of the invention have been heretofore described, it will be obvious to those having ordinary skill in the art that changes and modifications may be made thereto without departing from the scope and the spirit of the invention as hereinafter claimed.

What is claimed is:

1. A decorative air fragrance dispenser comprising:
an open container having a base portion and a perimetric wall portion continuous with the base portion;
a foliage anchoring device positioned within said container;
a foliage arrangement secured to said foliage anchoring device;
a sublimatable aromatic compound at least partially covering said foliage anchoring device and providing adhesion between the foliage anchoring device and said vessel.

2. The decorative air fragrance dispenser of claim 1, wherein said foliage anchoring device comprises a collapsible polymeric foam block.

3. The decorative air fragrance dispenser of claim 1, wherein said foliage anchoring device comprises a block of compressed wood particles that has been drilled or pierced to accept foliage stems.

4. The decorative air fragrance dispenser of claim 1, wherein said foliage anchoring device comprises multiple sandwiched layers of wire mesh.

5. The decorative air fragrance dispenser of claim 1, wherein said foliage anchoring device comprises a pinpoint holder.

6. The decorative air fragrance dispenser of claim 1, wherein said foliage arrangement includes artificial flowers.

7. The decorative air fragrance dispenser of claim 1, wherein said foliage arrangement includes dried natural flowers.

8. The decorative air fragrance dispenser of claim 1, wherein said sublimatable aromatic compound is a gel.

9. A decorative air fragrance dispenser comprising:
an open vessel;
an aromatic deodorizer compound having volatile components which partially fills the open vessel; and
a foliage arrangement consisting of multiple items, each of which has a stem that is partially embedded within the deodorizer compound.

10. The decorative air fragrance dispenser of claim 9, wherein at least some items of the foliage arrangement are selected from the group consisting of artificial flowers, dried natural flowers, cattails, and leaves.

11. The decorative air fragrance dispenser of claim 9, wherein each of the foliage arrangement items is attached to a foliage anchoring device, which is embedded with the deodorizer compound, said foliage anchoring device being selected from the group consisting of collapsible polymeric foam blocks, multiple sandwiched layers of wire mesh, blocks of wood or of compressed wood particles that have been drilled or pierced to accept foliage stems, and pinpoint holders.

12. A method of making a decorative air fragrance dispenser, said method comprising the steps of:
providing an open vessel of a desired size and shape;
providing a plurality of arrangeable floral items;
providing a floral arrangement holder;
providing a liquified aromatic deodorizer compound;
attaching each floral item to the floral arrangement holder;
placing the floral arrangement holder and attached floral items within said vessel;
pouring the liquified aromatic deodorizer compound into the vessel until the floral arrangement holder is at least partially submerged; and
allowing the liquified aromatic deodorizer compound to solidify.

13. The method of claim 12, wherein said aromatic deodorizer compound is a gel.

14. The method of claim 12, wherein said aromatic deodorizer compound has a melting point greater than about 40° C.

15. The method of claim 12, wherein at least some of the arrangeable floral items are selected from the group consisting of artificial flowers, dried natural flowers, cattails, and leaves.

16. The method of claim 12, wherein said said foliage anchoring device comprises a collapsible polymeric foam block.

17. The method of claim 16, wherein the said collapsible polymeric foam block is of an open-pore variety.

18. The decorative air fragrance dispenser of claim 12, wherein said floral arrangement holder comprises a block of compressed wood particles that has been drilled or pierced to accept foliage stems.

19. The decorative air fragrance dispenser of claim 12, wherein said floral arrangement holder comprises multiple sandwiched layers of wire mesh.

20. The decorative air fragrance dispenser of claim 12, wherein said floral arrangement holder comprises a pinpoint holder.
